(19) United States
(12) Patent Application Publication
(10) Pub. No.: US 2024/0342454 A1
(43) Pub. Date: Oct. 17, 2024

(54) MICRONEEDLE ARRAY WITH ANTISEPTICS

(71) Applicant: LTS LOHMANN THERAPIE-SYSTEME AG, Andernach (DE)

(72) Inventor: Markus Winterberg, Rheinbach (DE)

(21) Appl. No.: 18/579,663

(22) PCT Filed: Jul. 11, 2022
(86) PCT No.: PCT/EP2022/069218

§ 371 Date: Jan. 16, 2024

(30) Foreign Application Priority Data
Jul. 22, 2021 (DE) 10 2021 118 997.7

(51) Int. Cl. A61M 37/00; A61K 9/00; A61K 31/155; A61K 47/36

(52) U.S. Cl. CPC A61M 37/0015; A61K 9/0021; A61K 31/155; A61K 47/36; A61M 2037/0023; A61M 2037/0053

(57) ABSTRACT

The present invention relates to a microneedle array for use in the intradermal application of active substances, the array comprising a plurality of microneedles on a support which are formed of a formulation that contains a biodegradable polymer, an active substance and an antiseptic.

The present invention relates to a microneedle array for use in the intradermal application of active substances, which includes a plurality of microneedles on a support that are formed from a formulation containing a biodegradable polymer, an active substance, and an antiseptic.

The skin as the functionally most versatile organ of the human and animal organism consists of several layers and, in addition to its function as an enveloping organ, serves essential functions relating to metabolism, heat regulation, and immune response. The outermost skin layer, the so-called stratum corneum, has known barrier properties in order to prevent foreign substances from entering the body, and endogenous substances from leaving the body. The stratum corneum, which is a complex structure made of compacted keratotic cell remnants and has a thickness of approximately 10 to 30 micrometers, forms a water-tight membrane for protecting the body. The natural impermeability of the stratum corneum prevents the administration of most pharmaceutical agents and other substances through the skin within the scope of transdermal application.

Microneedle systems (MNS), consisting of a microneedle array (MNA) and optionally other components, have recently become established as an alternative for classical transdermal application, such as transdermal patches, of pharmaceutical and cosmetic agents. While transdermal patches are adhered to the skin and the released active substance is absorbed through the skin, microneedles, which are also referred to in part as skin penetration systems, are pressed against the skin by means of a compressive force from the array against the site of application to penetrate the stratum corneum, thereby generating a fluid channel, so that an active substance can be applied intradermally. Such microneedle arrays in Microneedle systems, the preparation thereof and the use thereof for administering a number of active substances are known in the prior art.

A survey of the recent developments in the field of microneedles is offered in the article “Microneedles as an alternative technology for transdermal drug delivery systems: a patent review” by M. L. Barreto Queiroz et al., issued in EXPERT OPINION ON THERAPEUTIC PATENTS 2020, vol. 30, No. 6, pp. 433-452. There are presented, among other things, the various systems, such as solid removable microneedles, coated microneedles, self-dissolving microneedles, hollow microneedles, and microneedles that form a hydrogel, wherein the concept of self-dissolving microneedles, in particular, has recently moved into the focus of attention.

Thus, WO 2019/115815 discloses a microneedle array for use in the intradermal application of a drug in the form of a salt, including a plurality of microneedles on a support, in which the microneedles comprise a formulation that contains at least one drug in the form of a salt and at least one biodegradable polymer.

In their article “Solid lipid nanoparticle-based dissolving microneedles; A promising intradermal lymph targeting drug delivery system with potential for enhanced treatment of lymphatic filariasis”, issued in Journal of Controlled Release 316 (2019) 34-52, A. D. Permana et al. describe the use of self-dissolving microneedles for the treatment of lymphatic filariasis.

WO 2019/092257 describes a microneedle array comprising a formulation of polyvinylpyrrolidone and HBsAg for use in intradermal application for hepatitis vaccination.

WO 2018/224559 describes a microneedle array comprising a formulation of polyvinylpyrrolidone and at least one glucagon-like peptide analogue for use in intradermal application for controlled release.

WO 2019/202170 relates to a microneedle array comprising a completely dissolvable formulation for use in the intradermal application of interferon, in which polyvinylpyrrolidone is the main component of the formulation.

Especially with microneedles provided for the administration of protein and nucleic acid drugs, there is a problem of providing a sterile product, because the currently approved sterilization methods, such as gamma sterilization, result in decomposition of the drugs to be administered. Therefore, obtaining sterile products requires expensive and complicated sterile manufacturing.

In order to address this problem, US 2017/0028184 proposes a microneedle system with in-situ anode and cathode, which is achieved by making the microneedles from a material that has an electrical potential. The metals used to prepare the microneedles are reported to have antiseptic properties.

EP 3 669 929 describes a microneedle array for plumping lips, wherein said microneedles may contain an antiseptic in addition to a water-soluble polymer.

US 2019/0358441 describes a microneedle patch, comprising a substrate, a microneedle matrix containing more than one microneedle, wherein each microneedle has a base, a cone-shaped sharp end filled with a mixture of a carrier bio-soluble material with an active component, and a plurality of wider cone-shaped branches between the sharp end and the base that are geometrically intersected with each other and are filled with a bio-soluble material, a base film connecting bases of the microneedles in the microneedle matrix with an inner surface and attached to the substrate with an outer surface, wherein the sharp end of the microneedle is placed on top of the branches of the microneedle, and wherein the substrate and the base film are made of flexible materials.

WO 2011/127149 discloses a method of controlled release of an effective amount of at least one or more bioactive or pharmaceutically active agents in a subject comprising administering a combination biomaterial to a subject, wherein the combination biomaterial comprises a combination biomaterial substrate and a degradable polymer wherein the degradable polymer comprises one or more bioactive or pharmaceutically active agents encapsulated by the degradable polymer, wherein the one or more bioactive agents or pharmaceutically active agents are delivered to the subject over a time period of greater than one week.

WO 2021/077119 provides organosilicon quaternary ammonium compounds, and their formulations for topical medical therapy in humans and animals.

Despite comprehensive efforts made in the prior art, when microneedles are used, there is still a residual risk of infection by introducing pathogens into the skin. Therefore, the object of the present invention is to provide a microneedle array that is capable of further reducing this risk and suitable for administering a high number of active ingredients.

Within the scope of the present invention, it has been surprisingly found that this object is achieved by a microneedle array in which the microneedles are formed from a co-formulation containing a biodegradable polymer and an antiseptic.

Therefore, the present invention firstly relates to a microneedle array for use in the intradermal application, including a plurality of microneedles on a support, wherein said microneedles are formed from a co-formulation containing a biodegradable polymer, an active substance, and an antiseptic having at least one guanide and/or biguanide group.

The microneedle array according to the invention has the advantage that an expensive sterile production of the microneedles can be dispensed with. Rather, despite being produced in a “low bioburden” environment, the microneedle array according to the invention have the necessary sterility to further minimize the risk of infection.

In contrast to solutions proposed in the prior art, i.e., to accommodate the antiseptic in cavities within the microneedles, the antiseptic of the invention is integrated and/or embedded in the polymer matrix of the microneedles, and immediately available. In addition, the solution according to the invention offers the advantage that cavities that may be provided in the microneedles can be used for accommodating further active substances rather than being blocked by the antiseptic.

The antiseptic is selected from the group of compounds from the class of guanides and biguanides, which, in addition to an advantageous incorporation into the polymer matrix of the biodegradable polymer, also exhibit a high compatibility with a variety of common drugs.

Within the scope of the present invention, an antiseptic is said to have a guanide group if the corresponding compound has the following structural element in its chemical structure:

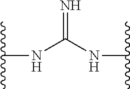 [SMILES: *NC(=N)N*]

An “antiseptic having a biguanide group” means those chemical compounds that have the following structural element:

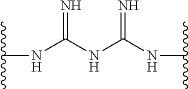 [SMILES: *NC(=N)NC(=N)N*]

Guanides and biguanides are known as antimicrobially active substances in the prior art. The guanides and biguanides can be employed as monomers or preferably as polymers, i.e., as a polyguanide and/or a polybiguanide. In a preferred embodiment, the guanides and/or biguanides are in the form of water-soluble, physiologically acceptable salts. More preferably, the guanides and biguanides are in the form of a hydrohalide, for example, hydrochloride or hydrobromide.

Particularly good results with respect to the compatibility with other drugs and an antiseptic activity were observed when a polybiguanide was employed as the antiseptic. Therefore, an embodiment of the present invention is preferred in which said antiseptic is a polybiguanide, preferably chlorhexidine and/or polyhexamethylenebiguanide (PHMB), especially polyhexamethylenebiguanide (PHMB).

The microneedle arrays according to the invention are provided, in particular, for the transdermal administration of cosmetically and pharmaceutically active substances, especially pharmaceutically active substances. They offer the advantage that said active substance is present together with the antiseptic and the biodegradable polymer as a homogeneous formulation from which the microneedles are made. The active substance and the antiseptic are released by dissolving the polymer, and are thus available directly at the site of administration.

Therefore, said active substance, especially a medicament, is integrated or incorporated in the microneedles, or in the matrix of formulation of the microarray. Further, at least one active substance, especially medicament, can be applied to the microneedles or support. However, it is preferred that said active substance, especially medicament, is a component of the microneedles and for that purpose is integrated or incorporated in the microneedles, especially in the tip of the microneedles.

Active substances employed according to the invention include, in particular, medicaments as defined in the EU directive 2001/83/EG (Community code relating to medicinal products for human use).

Within the scope of the present invention, it has been found that the microneedle arrays according to the invention are suitable for administering a variety of active substances. Thus, in first experiments, a decrease in drug activity could not be observed. Preferably, the active substance is a pharmaceutically active substance. Preferably, the active substance is selected from the group consisting of analgetics, anaesthetic agents, antiasthmatic agents, antibiotics, antidepressives, antidiabetic agents, antimycotics, antihypertensives, anti-inflammatory agents, antineoplastics, anxiolytics, nucleic acids, immunostimulant substances, Immunosuppressives, vitamins, hormones, peptides, proteins, and vaccines. In particular, said active substance is a vaccine, an analgetic, or insulin. Even more preferably, the active substance is a vaccine.

In a further preferred embodiment, said active substance is a nucleic acid, especially mRNA and silencer mRNA.

In a preferred embodiment, said active substance is in the form of lipid nanoparticles. The active substance may be employed individually, or in a combination of several active substances.

In a preferred embodiment, the microneedles have a multilayer structure, in which each of the layers is formed from a biodegradable polymer. Preferably, at least one layer is formed from a formulation that contains a biodegradable polymer, an active substance, and an antiseptic. In a particularly preferred embodiment, this layer forms the outermost layer of the microneedles. In a preferred embodiment, at least one of the inner layers is formed from a formulation that contains a biodegradable polymer, and an antiseptic. In this manner, a sustained release of the antiseptic can be reached.

In another, alternatively preferred embodiment, the outermost layer of the microneedles is formed from a formulation that contains a biodegradable polymer and an antiseptic. In this manner, the antiseptic can display its activity at the site of administration even before the active substance is released.

The microneedles according to the present invention are made of a formulation that contains a biodegradable polymer. More preferably, the microneedles are made of a co-formulation of a biodegradable polymer, an active substance, and an antiseptic. In practical application, those microneedle arrays that will dissolve by themselves have proven advantageous, in particular. In particular, these are microneedle arrays that remain in the skin after administration, and become dissolved with release of the active substance. Since the microneedles remain in the skin, a short application time can be achieved, whereby irritations of the skin's surface induced by the support material can be avoided, for example. Further, this method enables an immediate and constant release of active substances. For the microneedles to be suitable for remaining in the skin, the materials used for preparing them must be toxicologically safe on the one hand, and self-dissolving on the other. This combination of properties is achieved, in particular, when the microneedles are made of certain biodegradable polymers.

Therefore, an embodiment is preferred in which said biodegradable polymer is selected from the group consisting of polyvinylpyrrolidone, polyvinyl alcohols, cellulose, dextrans, glycans, glycosaminoglycans, hyalurones, alpha-hydroxy acids, such as lactic acid and/or glycolic acid, polylactides, polyglycolides, poly(lactide-co-glycolide), and copolymers thereof with polyethylene glycols, polyanhydrides, poly(ortho) esters, polyurethanes, polybutyric acids, polyvaleric acids, and poly(lactide-co-caprolactone) s. In a particularly preferred embodiment, said biodegradable polymer is selected from polyvinylpyrrolidones, and dextrans.

The microneedle array can have a large number of microneedles in order to deliver a substance through or into a patient's skin, wherein said microneedle array is applied to the patient's skin. Each of the microneedles of the microneedle array preferably has a lengthy shaft with two ends, where one end of the shaft is the base of the microneedle with which the microneedle is attached to the sheet-like support, or with which the microneedle is integrated into the sheet-like support. The end of the shaft opposite the base preferably has a tapered design, in order to enable the microneedle to penetrate the skin as easily as possible. A hollow microneedle may have at least one passage or channel or at least one bore that extends from the base of the microneedle to the tip of the microneedle or to approximately the tip of the microneedle. The passages preferably have a round cross-section.

The microneedles may have a shaft with a round cross-section or with a cross-section that is not round, for example, a triangular, quadrangular or polygonal cross-section. The shaft may have a passage or several passages that go from the needle base to the needle tip, or approximately to the needle tip. The microneedles may be formed as hooks (or barbs), wherein one or more of such microneedles have one or more of such hooks. Further, the microneedles may have a helical shape and a rotatable mount, which facilitates penetration into the skin when a rotating movement is applied, and cause anchoring within the skin, as described, for example, in DE 103 53 629 A1, especially in the desired penetration depth in the epidermis.

The diameter of a microneedle is usually from 1 μm to 500 μm, preferably from 10 μm to 100 μm. The diameter of a passage is usually from 3 μm to 80 μm and is suitable for the passing of, preferably liquid, materials, solutions and preparations. The length of a microneedle is usually from 10 μm to 1000 μm, especially from 100 μm to 500 μm.

The microneedles are attached to a sheet-like support through their base, or integrated in a sheet-like support. The microneedles are preferably provided to stand essentially perpendicularly to the surface of the support. The microneedles may have a regular or an irregular arrangement. An array of several microneedles may have microneedles with different cross-sectional shapes, differently sized diameters, and/or different lengths. Said array of several microneedles may exclusively have hollow microneedles. The array may both comprise solid microneedles, and have partly solid composites, such as solid microneedles permeated by liquid inclusions.

The microneedle array may have a sheet-like support, wherein said support essentially has a disk-like, plate-like or sheet-like basic shape. The support may have a circular, oval, triangular, quadrangular or polygonal basic area. The support may be made of different materials, for example, a metal, a ceramic material, a semiconductor, an organic material, a polymer, or a composite. As materials that are suitable for preparing the support, preferably, there may be mentioned sheets or sheet-like materials, for example, microporous membranes, preferably made of polyethylene (PE) or polypropylene (PP), or diffusion membranes, preferably made of ethylene/vinyl acetate copolymer (EVA) or polyurethane (PU). Suitable materials for preparing the support may be selected from the group consisting of polyesters, such as polyethylene terephthalates (PET), polycarbonates (PC), polyetherketones (PEK), polyethylene naphthalate (PEN), polybutylene terephthalates (PBT), polyurethanes (PU), polystyrenes (PS), Polyamides (PA), polyoxymethylene (POM), polyolefins, such as polyethylene (PE) and polypropylene (PP), polytetrafluoroethylene (PTFE), polyvinyl chloride (PVC), polyvinylidene chloride (PVDC), polylactate (PLA), and cellulose-based polymers, such as cellulose hydrate or cellulose acetate. Suitable materials for preparing the support may also be selected from the group of metals consisting of aluminum, iron, copper, gold, silver, platinum, alloys of the above mentioned metals, and other pharmaceutically acceptable metal foils, or sheets coated with vapor-deposited metals.

Preferably, the support is made of a flexible material, for example, a plastic. A support made of a flexible material can better conform to the skin surface and its curvature as compared to a support made of a non-flexible material. This achieves a better contact of the microneedle array with the skin, whereby the reliability of the microneedle array is improved.

In a preferred embodiment, the support may also be provided with an antiseptic.

The present invention further relates to a process for preparing said microneedle array according to the invention, which is, in particular, characterized in that the microneedles are made, especially consist, of a biodegradable polymer, in which an antiseptic is embedded into the polymer matrix.

The process according to the invention includes the following steps:
a) providing a liquid formulation comprising a biodegradable polymer, an active substance, and an antiseptic with at least one guanide and/or biguanide group;
b) filling the formulation into a flexible mold that corresponds to a negative print of the microneedle array to be prepared;
c) drying the formulation in the mold; and
d) demolding the dried microneedle array.

In a preferred embodiment, the biodegradable polymer and/or the antiseptic is in the form of its monomers in the provided liquid formulation.

Said drying of the formulation in the flexible molds to form the microneedle array may be effected in a way known to those skilled in the art, but preferably by heating and/or ventilation.

The antiseptic in the formulation is preferably in an amount that enables incorporation into the polymer matrix, while it achieves a sufficient antiseptic effect to minimize the risk of an infection by the microneedles. In a preferred embodiment, the amount of antiseptic in the liquid formulation is from 100 to 600 μg, preferably from 150 to 550 μg, especially 200 μg or 400 μg, respectively based on 1 ml of the liquid formulation.

In a preferred embodiment, further formulations may be introduced into the flexible mold to form a multilayer structure.

The present invention further relates to the use of a formulation containing a biodegradable polymer, an active substance, and an antiseptic with at least one guanide and/or biguanide group for preparing microneedle arrays, especially microneedle arrays for transdermal application.

The present invention is further explained by means of the following Examples, which should by no means be understood as limiting the idea of the invention.

### EXAMPLE

The antibacterial activity of the microneedle array according to the invention was tested by means of an inhibition zone test. Thus, a microneedle array was prepared from a formulation containing dextran as the biodegradable polymer, and 200 μg/ml of PHMB, and applied to an agar plate with bacteria. After an incubation period of 24 hours, the formation of an inhibition zone as a measure of the reduction of bacterial growth around the microneedle array can be clearly seen, wherein the contact sites of the microarrays are respectively marked with white dots. Surprisingly, the same results were also obtained with microneedle arrays that had been stored already for one month.

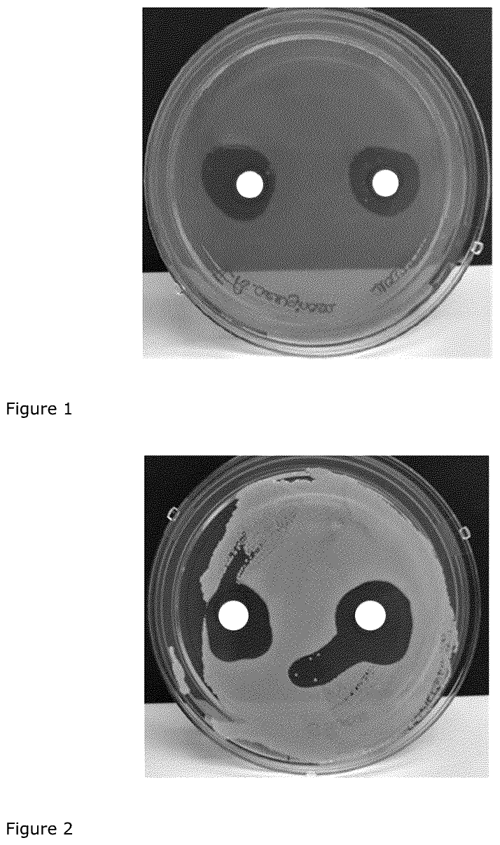

FIG. 1 shows the antibacterial effect of the microneedle array according to the invention towards Pseudomonas aeruginosa.

FIG. 2 shows the antibacterial effect of the microneedle array according to the invention towards Staphylococcus aureus.

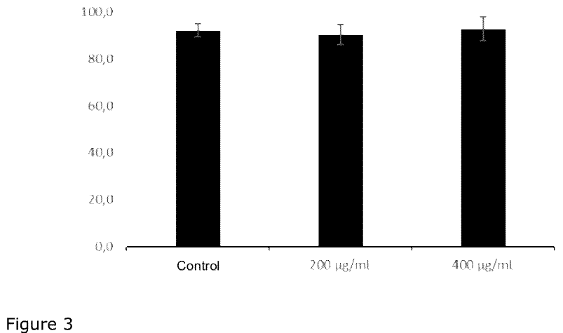

Further, the influence of the antiseptic on other active substances contained in the formulation was tested, wherein the hepatitis B S-antigen (HBsAg) was used as an exemplary active substance, and PHMB was used as the antiseptic. From the consideration that, if the antiseptic should adversely affect the integrity of HBsAg, this should manifest itself in a reduced binding of the drug in an HBsAg-specific ELISA, microneedle arrays according to the invention were prepared from a formulation of dextran and 200 μg/ml or 400 μg/ml of PHMB and 20 μmol of HBsAg per microarray, and measured. An HBsAg ELISA kit from Alpha Diagnostic was used as the ELISA. The results are summarized in FIG. 3, which shows the two microarrays according to the invention with 200 μg/ml and 400 μg/ml of PHMB, respectively, and the control test series without PHMB. The lower the amount found in the ELISA, the larger is the influence of the antiseptic on the active substance. As can be seen from the data in FIG. 3, no significant difference in antigen binding can be seen between the control and the PHMB-containing specimens.

1. A microneedle array including a plurality of microneedles on a support, characterized in that said microneedles are formed from a co-formulation containing a biodegradable polymer, an active substance, and an antiseptic having at least one guanide and/or biguanide group.

2. The microneedle array according to claim 1, characterized in that said antiseptic is a polybiguanide, preferably chlorhexidine and/or polyhexamethylenebiguanide (PHMB), especially polyhexamethylenebiguanide (PHMB).

3. The microneedle array according to claim 1, characterized in that said active substance is selected from the group consisting of analgetics, anaesthetic agents, antiasthmatic agents, antibiotics, antidepressives, antidiabetic agents, antimycotics, antihypertensives, anti-inflammatory agents, antineoplastics, anxiolytics, nucleic acids, immunostimulant substances, Immunosuppressives, vitamins, hormones, peptides, proteins, and vaccines.

4. The microneedle array according to claim 1, characterized in that said active substance is in the form of enveloped lipid nanoparticles.

5. The microneedle array according to claim 1, characterized in that said biodegradable polymer is selected from the group consisting of polyvinylpyrrolidone, polyvinyl alcohols, cellulose, dextrans, glycans, glycosaminoglycans, hyalurones, alpha-hydroxy acids, such as lactic acid and/or glycolic acid, polylactides, polyglycolides, poly(lactide-co-glycolide), and copolymers thereof with polyethylene glycols, polyanhydrides, poly(ortho) esters, polyurethanes, polybutyric acids, polyvaleric acids, and poly(lactide-co-caprolactone) s.

6. A process for preparing a microneedle array comprising the steps of:
a) providing a liquid formulation comprising a biodegradable polymer, an active substance, and an antiseptic with at least one guanide and/or biguanide group;
b) filling the formulation into a flexible mold that corresponds to a negative print of the microneedle array to be prepared;
c) drying the formulation in the mold; and
d) demolding the dried microneedle array.

7. The process according to claim 6, characterized in that the amount of antiseptic in the liquid formulation is from 100 to 600 μg based on 1 ml of the formulation.

8. A formulation containing a biodegradable polymer, an active substance, and an antiseptic with at least one guanide and/or biguanide group, the formulation being molded into microneedle arrays.

9. The process of claim 6, wherein the microneedle array includes a plurality of microneedles on a support, characterized in that said microneedles are formed from the biodegradable polymer, the active substance, and the antiseptic having at least one guanide and/or biguanide group.

10. The formulation of claim 8, wherein the microneedle array includes a plurality of microneedles on a support, characterized in that said microneedles are formed from the biodegradable polymer, the active substance, and the antiseptic having at least one guanide and/or biguanide group.

11. The formulation of claim 8, characterized in that the amount of antiseptic in the liquid formulation is from 100 to 600 μg.

12. The microneedle array of claim 1, characterized in that the amount of antiseptic is from 100 to 600 μg.

13. The microneedle array according to claim 2, characterized in that said active substance is selected from the group consisting of analgetics, anaesthetic agents, antiasthmatic agents, antibiotics, antidepressives, antidiabetic agents, antimycotics, antihypertensives, anti-inflammatory agents, antineoplastics, anxiolytics, nucleic acids, immunostimulant substances, Immunosuppressives, vitamins, hormones, peptides, proteins, and vaccines.

14. The microneedle array according to claim 2, characterized in that said active substance is in the form of enveloped lipid nanoparticles.

15. The microneedle array according to claim 2, characterized in that said biodegradable polymer is selected from the group consisting of polyvinylpyrrolidone, polyvinyl alcohols, cellulose, dextrans, glycans, glycosaminoglycans, hyalurones, alpha-hydroxy acids, such as lactic acid and/or glycolic acid, polylactides, polyglycolides, poly(lactide-co-glycolide), and copolymers thereof with polyethylene glycols, polyanhydrides, poly(ortho) esters, polyurethanes, polybutyric acids, polyvaleric acids, and poly(lactide-co-caprolactone) s.

16. The microneedle array of claim 2, characterized in that the amount of antiseptic is from 100 to 600 μg.

17. The microneedle array according to claim 3, characterized in that said active substance is in the form of enveloped lipid nanoparticles.

18. The microneedle array according to claim 3, characterized in that said biodegradable polymer is selected from the group consisting of polyvinylpyrrolidone, polyvinyl alcohols, cellulose, dextrans, glycans, glycosaminoglycans, hyalurones, alpha-hydroxy acids, such as lactic acid and/or glycolic acid, polylactides, polyglycolides, poly(lactide-co-glycolide), and copolymers thereof with polyethylene glycols, polyanhydrides, poly(ortho) esters, polyurethanes, polybutyric acids, polyvaleric acids, and poly(lactide-co-caprolactone) s.

19. The microneedle array of claim 3, characterized in that the amount of antiseptic is from 100 to 600 μg.

20. The microneedle array according to claim 4, characterized in that said biodegradable polymer is selected from the group consisting of polyvinylpyrrolidone, polyvinyl alcohols, cellulose, dextrans, glycans, glycosaminoglycans, hyalurones, alpha-hydroxy acids, such as lactic acid and/or glycolic acid, polylactides, polyglycolides, poly(lactide-co-glycolide), and copolymers thereof with polyethylene glycols, polyanhydrides, poly(ortho) esters, polyurethanes, polybutyric acids, polyvaleric acids, and poly(lactide-co-caprolactone) s.
